(19) United States
(12) Patent Application Publication
(10) Pub. No.: US 2023/0270553 A1
(43) Pub. Date: Aug. 31, 2023

(54) IMPLANTABLE INFLATABLE DEVICE AND THERAPEUTIC METHODS

(71) Applicant: Boston Scientific Scimed, Inc., Maple Grove, MN (US)

(72) Inventors: Daragh Nolan, Via Youghal (IE); John Gildea, Kilcock (IE); Thomas Sinnott, Wexford (IE); Brian P. Watschke, Minneapolis, MN (US); Natalie A. Borgos, Roseville, MN (US); Noel Smith, Windgap (IE)

(21) Appl. No.: 18/155,534

(22) Filed: Jan. 17, 2023

Related U.S. Application Data
(60) Provisional application No. 63/266,918, filed on Jan. 19, 2022.

(51) Int. Cl. A61F 2/26; A61F 2/48

(52) U.S. Cl. CPC A61F 2/26; A61F 2/482; A61F 2250/0013

(57) ABSTRACT

An implantable inflatable device includes a fluid reservoir defining a cavity, an inflatable member, a pump assembly configured to transfer fluid from the fluid reservoir to the inflatable member, and a programmable control module, the programmable control module being configured activate the pump assembly to transfer fluid from the fluid reservoir to the inflatable member.

### CROSS-REFERENCE TO RELATED APPLICATION

This application claims priority to U.S. Provisional Patent Application No. 63/266,918, filed on Jan. 19, 2022, entitled “IMPLANTABLE INFLATABLE DEVICE AND THERAPEUTIC METHODS”, the disclosure of which is incorporated by reference herein in its entirety.

### TECHNICAL FIELD

This disclosure relates generally to bodily implants, and more specifically to bodily implants including an inflatable member, a fluid reservoir, and a pump.

### BACKGROUND

Implantable inflatable devices often include one or more pumps that regulate a flow of fluid between different portions of the implantable device to provide for inflation and deflation of one or more fluid fillable implant components of the device. For example, some implantable inflatable devices include an inflatable member, a fluid reservoir, and a pump or pump assembly. In such implantable inflatable devices, it may be desirable to inflate or deflate or inflate and deflate the inflatable member according to a schedule. Accordingly, there is a need for an implantable inflatable device that is configured to inflate or deflate or inflate and deflate a plurality of times according to a prescribed schedule.

### SUMMARY

According to an aspect, an implantable inflatable device includes a fluid reservoir defining a cavity, an inflatable member, a pump assembly configured to transfer fluid from the fluid reservoir to the inflatable member, and a programmable control module, the programmable control module being configured activate the pump assembly to transfer fluid from the fluid reservoir to the inflatable member.

In some embodiments, the programmable control module is configured to be controlled by a device located outside of the body of the patient.

In some embodiments, the programmable control module is configured to activate the pump assembly according to a schedule. In some embodiments, the programmable control module is configured to activate the pump assembly while the user is asleep. In some embodiments, the programmable control module is configured to activate the pump assembly such that the inflatable member inflates and deflates according to a schedule. In some embodiments, the programmable control module is configured to activate the pump assembly such that the inflatable member inflates and deflates while the user is asleep. In some embodiments, the programmable control module is configured to activate the pump assembly such that the inflatable member inflates and deflates according to a schedule.

In some embodiments, the programmable control module is configured to activate the pump assembly such that the inflatable member inflates and deflates a plurality of times over a period of several hours. In some embodiments, the programmable control module is configured to activate the pump assembly such that the frequency of the pump actuation pulses over a period of time. In some embodiments, the programmable control module is configured to activate the pump assembly such that the frequency of the pump actuation pulses a plurality of time over a period of 10 minutes.

In some embodiments, the inflatable member is configured to be disposed within a pelvic region of a patient. In some embodiments, the inflatable member is configured to be at least partially disposed within a penis of a patient.

In some embodiments, the device includes a tubular member operatively connecting the fluid reservoir and the pump assembly. In some embodiments, a tubular member operatively connecting the inflatable member and the pump assembly.

In some embodiments, the device includes a first tubular member operatively connecting the fluid reservoir and the pump assembly and a second tubular member operatively connecting the pump assembly and the inflatable member.

In some embodiments a method includes activating a pump assembly of an inflation device disposed within a body of a user such that a pressure within an inflatable member of the inflation device increases and decreases a plurality of times according to a schedule.

In some embodiments, the pressure within the inflatable member of the inflation device increases and decreases a plurality of times while the user is asleep. In some embodiments, the pressure within an inflatable member of the inflation device increases and decreases a plurality of times over a period of hours. In some embodiments, the pressure within an inflatable member of the inflation device increases and decreases a plurality of times over a period of minutes.

In some embodiments, the inflation device is at least partially disposed within a pelvic region of the user. In some embodiments, the inflation device is at least partially disposed within a penis of a user. In some embodiments, the inflatable member of the inflation device is at least partially disposed within a pelvic region of the user. In some embodiments, the inflation device is at least partially disposed within a penis of a user. In some embodiments, the inflation device includes a fluid reservoir. In some embodiments, the inflation device includes a programmable control module, the programmable control module being configured activate the pump assembly.

### BRIEF DESCRIPTION OF THE DRAWINGS

FIG. 1 is a schematic illustration of an implantable inflatable device according to an aspect.

FIG. 2 is a perspective view of an implantable inflatable device according to an aspect.

FIG. 3 is a schematic illustration of a pump assembly of the implantable inflatable device of FIG. 2.

FIG. 4 is a graph showing the pressure of the inflatable member at different times.

FIG. 5 is a cross-sectional view of a penis of a patient.

FIG. 6 is a cross-sectional view of a penis of a patient having a portion of an implantable inflatable device disposed therein.

FIG. 7 is a graph showing the frequency of the pump actuation at different times.

### DETAILED DESCRIPTION

Detailed implementations are disclosed herein. However, it is understood that the disclosed implementations are merely examples, which may be embodied in various forms. Therefore, specific structural and functional details disclosed herein are not to be interpreted as limiting, but merely as a basis for the claims and as a representative basis for teaching one skilled in the art to variously employ the implementations in virtually any appropriately detailed structure. Further, the terms and phrases used herein are not intended to be limiting, but to provide an understandable description of the present disclosure.

The terms “a” or “an,” as used herein, are defined as one or more than one. The term “another,” as used herein, is defined as at least a second or more. The terms “including” and/or “having”, as used herein, are defined as comprising (i.e., open transition). The term “coupled” or “moveably coupled,” as used herein, is defined as connected, although not necessarily directly and mechanically.

In general, the implementations are directed to bodily implants. The term patient or user may hereinafter be used for a person who benefits from the medical device or the methods disclosed in the present disclosure. For example, the patient can be a person whose body is implanted with the medical device or the method disclosed for operating the medical device by the present disclosure.

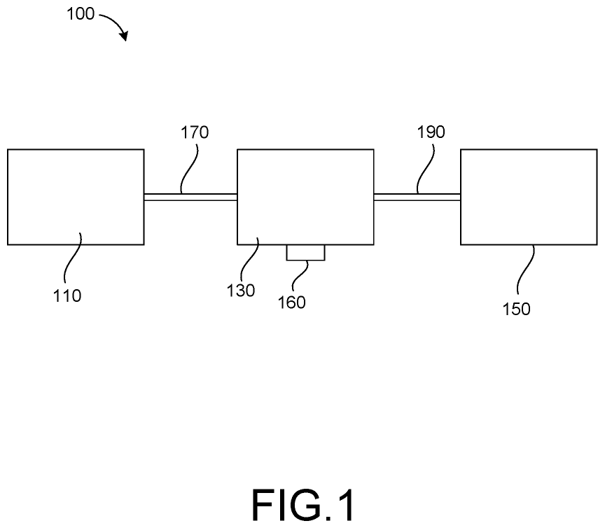

FIG. 1 is a schematic illustration of an implantable inflatable device 100. The device 100 includes a fluid reservoir 110, a pump assembly 130, and an inflatable member 150. The fluid reservoir 110 is operatively or fluidically coupled to the pump assembly 130 via connection member 170. The connection member 170 may be a tubular member such as a kink resistant tubing (KRT). In other implementations, the fluid reservoir 110 is operatively or fluidically coupled to the pump assembly 130 via a different mechanism. Similarly, the inflatable member 150 is operatively or fluidically coupled to the pump assembly 130 via connection member 190. Connection member 190 may be a tubular member such as a kink resistant tubing (KRT). In other implementations, the inflatable member 150 is operatively or fluidically coupled to the pump assembly 130 via a different mechanism.

The implantable inflatable device 100 may be configured to be implanted into a body of a patient or user. For example, in some embodiments, the implantable inflatable device 100 is a penile implant. In such embodiments, the inflatable member 150 may be implanted into the corpus cavernosae of the patient or user, the fluid reservoir 110 may be implanted in the abdomen or pelvic cavity of the user (e.g., the fluid reservoir 110 may be implanted in the lower portion of the user's abdominal cavity or the upper portion of the user's pelvic cavity), and the pump assembly 130 may be implanted into a portion of the body of the user, such as an abdomen of the user. In other embodiments, the implantable inflatable device 100 is implanted into a different portion of the body of the patient and/or is implanted for a different purpose. For example, in some embodiments, the implantable inflatable device 100 may be an artificial sphincter, such as an artificial urinary sphincter.

The pump assembly 130 may include a pump or more than one pump that is configured to pump fluid into the inflatable member 150 during an inflation cycle. In some examples, the pump or pumps may be mechanically and/or programmatically controlled by a controller. For example, in the illustrated embodiment, the device 100 includes a programmable control module 160.

The inflatable member 150 may be capable of expanding upon the injection of fluid into a cavity of the inflatable member 150. For instance, upon injection of the fluid into the inflatable member 150, the inflatable member 150 may increase its length and/or width, as well as increase its rigidity. In some examples, the inflatable member 150 may include a pair of inflatable cylinders or at least two cylinders, e.g., a first cylinder member and a second cylinder member. The volumetric capacity of the inflatable member 150 may depend on the size of the inflatable cylinders.

The fluid reservoir 110 may include a container having an internal cavity or chamber configured to hold or house fluid that is used to inflate the inflatable member 150. The volumetric capacity of the fluid reservoir 110 may vary. In some examples, the volumetric capacity of the fluid reservoir 110 may be 3 to 150 cubic centimeters. In some examples, the fluid reservoir 110 is constructed from the same material as the inflatable member 150. In other examples, the fluid reservoir 110 is constructed from a different material than the inflatable member 150. In some examples, the fluid reservoir 110 contains a larger volume of fluid than the inflatable member 150.

In the illustrated embodiment, the programmable control module 160 is operatively coupled to the pump assembly 130. In some embodiments, the programmable control module 160 is configured to activate and deactivate the pump or pumps. Accordingly, the programmable control module 160 is configured to activate or deactivate the pump or pumps in order to control the pressure or the state (inflated state or deflated state) of the inflatable member 150.

In some embodiments, the programmable control module 160 may be programed to activate or deactivate the pump or pumps according to a schedule. For example, in some embodiments, the programmable control module 160 is configured to cause the inflatable member 150 to inflate and deflate while the user is asleep. In some embodiments, the implant 100 includes an accelerometer. The accelerometer may be configured to determine when the patient is asleep. In other embodiments, the implant 100 includes another means or mechanism for determining when the patient is asleep. In some embodiments, the programmable control module 160 is configured to cause the inflatable member 160 to inflate and deflate a plurality of times over a period of a few hours.

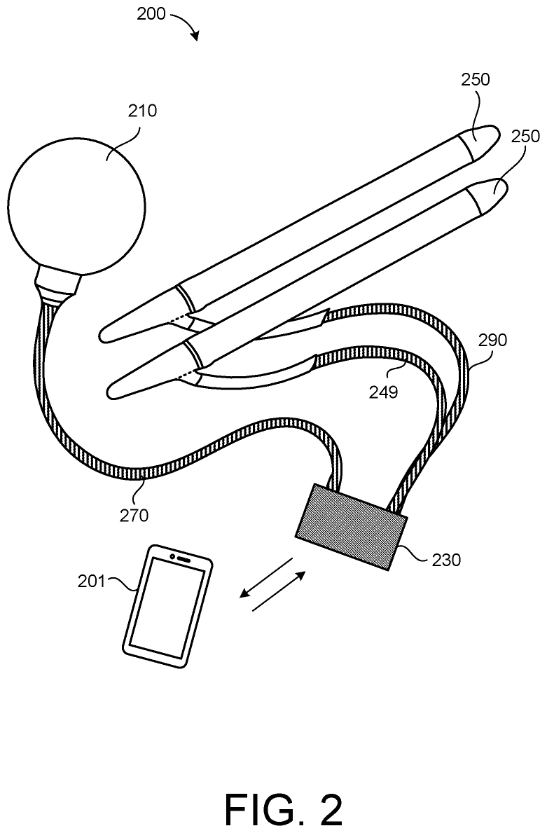

FIG. 2 illustrates an inflatable penile prosthesis 200 having a pump assembly 230 according to an aspect. The pump assembly 230 may include valves and may include manually actuated pump bulb or may include an electronically controlled pump. The penile prosthesis 200 may include one or more inflatable members or inflatable cylinders 250. In the illustrated embodiment, the prosthesis 200 includes a pair of inflatable cylinders 250. The inflatable cylinders 250 are configured to be implanted in a penis. For example, one of the inflatable cylinders 250 may be disposed on one side of the penis, and the other inflatable cylinder 250 may be disposed on the other side of the penis. Each inflatable cylinder 250 may include a first end portion, a cavity or inflation chamber, and a second end portion having a rear tip. The first end portion of the inflatable cylinder 250 may be at least partially disposed within the crown portion of the penis. The second end portion may be implanted into the patient's pubic region with the rear tip proximate the pubic bone.

The pump assembly 230 may be implanted into the body of the patient. In some embodiments, the pump assembly 230 may be implanted into an abdomen of the patient. A pair of conduit connectors 290 may attach the pump assembly 230 to the inflatable cylinders 250 such that the pump assembly 230 is in fluid communication with the inflatable cylinders 250. Also, the pump assembly 230 may be in fluid communication with a fluid reservoir 210 via a connection member or a conduit connector 270. The fluid reservoir 210 may be implanted into the user's abdomen.

In order to implant the inflatable cylinders 250, the surgeon first prepares the patient. The surgeon often makes an incision in the penoscrotal region, e.g., where the base of the penis meets with the top of the scrotum. From the penoscrotal incision, the surgeon may dilate the patient's corpus cavernosae to prepare the patient to receive the inflatable cylinders 250. The corpus cavernosum is one of two parallel columns of erectile tissue forming the dorsal part of the body of the penis, e.g., two slender columns that extend substantially the length of the penis. The surgeon will also dilate two regions of the pubic area to prepare the patient to receive the second end portion. The surgeon may measure the length of the corpora cavernosae from the incision and the dilated region of the pubic area to determine an appropriate size of the inflatable cylinders 250 to implant.

After the patient is prepared, the penile prosthesis 200 is implanted into the patient. The tip of the first end portion of each inflatable cylinder 250 may be attached to a suture. The other end of the suture may be attached to a needle member (e.g., Keith needle). The needle member is inserted into the incision and into the dilated corpus cavernosum. The needle member is then forced through the crown of the penis. The surgeon tugs on the suture to pull the inflatable cylinder 250 into the corpus cavernosum. This is done for each inflatable cylinder 250 of the pair. Once the inflation chamber is in place, the surgeon may remove the suture from the tip. The surgeon then inserts the second end portion. The surgeon inserts the rear end of the inflatable cylinder 250 into the incision and forces the second end portion toward the pubic bone until each inflatable cylinder 250 is in place.

In some embodiment, the pump assembly 230 includes an electric pump or pump system.

A user may use an external device 201 to control the inflatable penile prosthesis 200. In some examples, the user may use the external device 201 to inflate or deflate the inflatable cylinders 250. For example, in response to the user activating an inflation cycle using the external device 201, the external device 201 may transmit a wireless signal to the electronic pump assembly 206 or to the programmable control module to initiate the inflation cycle to transfer fluid from the fluid reservoir 210 to the inflatable cylinders 250. In some examples, in response to the user activating a deflation cycle using the external device 201, the external device 201 may transmit a wireless signal to the electronic pump assembly 206 or to the programmable control module to initiate the deflation cycle to transfer fluid from the inflatable cylinders 250 to the fluid reservoir 210. In some examples, during the deflation cycle, fluid is transferred back until the pressure in the inflatable cylinders 250 reaches a partial inflation pressure.

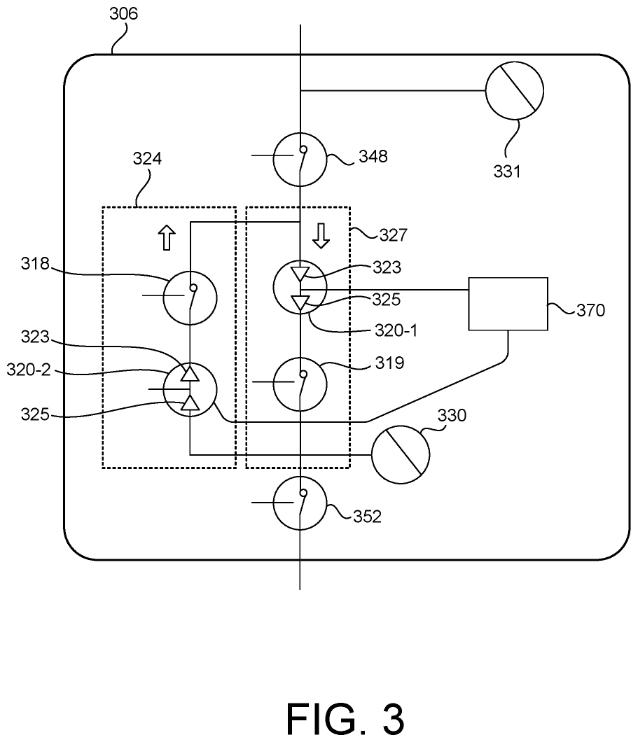

FIG. 3 illustrates an example of a portion of an electronic pump assembly 306 and a programmable control module 370 according to an aspect. The electronic pump assembly 306 may be an example of the electronic pump assembly 130 of FIG. 1 and/or the electronic pump assembly 230 of FIG. 2 and may include any of the details discussed with reference to the device 100 of FIG. 1 and/or the device 200 of FIG. 2.

The electronic pump assembly 306 is configured to transfer fluid between the fluid reservoir and the inflatable member. The electronic pump assembly 306 may automatically transfer fluid between the fluid reservoir and the inflatable member without the user manually operating a pump (e.g., squeezing and releasing a pump bulb).

The electronic pump assembly 306 includes a pump 320-1 disposed within a fluid passageway 327 (e.g., a fill passageway), and an active valve 318 disposed within a fluid passageway 324 (e.g., an empty passageway). The pump 320-1 may be an electromagnetic pump or a Piezoelectric pump. The pump 320-1 may include a passive check valve 323 and a passive check valve 325. The fluid passageway 327 may be a fluid branch that is separate (and parallel) to the fluid passageway 324. The fluid passageway 327 is the passageway that transfers fluid from the fluid reservoir to the inflatable member. The fluid passageway 324 is the passageway that transfers fluid from the inflatable member to the fluid reservoir. The pump 320-1 is disposed in parallel with the active valve 318.

In some examples, the electronic pump assembly 306 may include an active valve 319 in series with the pump 320-1 (e.g., the pump 320-1 and the active valve 319 are disposed within the fluid passageway 327). In some examples, the electronic pump assembly 306 may include a pump 320-2 in series with the active valve 318 (e.g., the pump 320-2 and the active valve 318 are disposed in the fluid passageway 324). The pump 320-2 may be an electromagnetic pump or a Piezoelectric pump. The pump 320-2 may include a passive check valve 323 and a passive check valve 325. In some examples, the electronic pump assembly 306 includes an active valve 348 that is fluidly connected to the fluid reservoir. The active valve 348 may be in series with either the active valve 318 (and the pump 320-2) or the pump 320-1 (and the active valve 319). In some examples, the electronic pump assembly 306 includes an active valve 352 that is fluidly connected to the inflatable member. The active valve 352 may be in series with either the active valve 319 (and the pump 320-1) or the pump 320-2 (and the active valve 318).

The active valve 348, the pump 320-1, the active valve 318, the active valve 352, the active valve 318, and the pump 320-2 may be electronically controlled by a controller and/or driver. The pump 320-1 and the pump 320-2 may be unidirectional or bidirectional. With respect to the fluid passageway 327, in some examples, the pump 320-1 and the active valve 319 may swap positions (e.g., where the active valve 319 is in series between the active valve 348 and the pump 320-1). With respect to the fluid passageway 324, in some examples, the active valve 318 and the pump 320-2 may swap positions (e.g., where the pump 320-2 is in series with and between the active valve 318 and the active valve 348).

In some examples, one or more additional active valves and/or one or more additional pumps are disposed in series within the fluid passageway 327. In some examples, one or more additional active valves and/or one or more additional pumps are disposed in series within the fluid passageway 324. In some examples, the electronic pump assembly 306 may include one or more additional (and parallel) fluid passageways, where each additional (and parallel) fluid passageway may include one or more active valves and one or more pumps.

In some examples, the electronic pump assembly 306 may include a pressure sensor 330 and a pressure sensor 331. The pressure sensor 330 and the pressure sensor 331 are connected to a controller, where the controller receives the measured pressure from the pressure sensor 330 and the pressure sensor 331.

The pressure sensor 330 is configured to measure the pressure in the device. For example, in some embodiments the pressure sensor 330 may be configured to measure the pressure in the inflatable member. In other embodiments, the pressure sensor may be configured to measure the pressure of other portions of the device. The controller may receive the measured pressure from the pressure sensor 330 and automatically control the active valves and/or the pump to regulate the pressure. In some examples, the pressure sensor 331 is configured to measure the pressure in the fluid reservoir. In some examples, the pressure sensor 331 may detect intra-abdominal pressure (which can increase during activities such as exercise, and the controller can control the active valves and pump to minimize or prevent accidental inflations. In some examples, the electronic pump assembly 306 may include one or more pressure sensors at other locations within the electronic pump assembly 306. For example, a pressure sensor may be disposed between the active valve 348 and the pump 320-1. In some examples, a pressure sensor may be disposed between the pump 320-1 and the active valve 319. In some examples, a pressure sensor may be disposed between the active valve 348 and the active valve 318. In some examples, a pressure sensor may be disposed between the active valve 318 and the pump 320-2.

In the illustrated embodiment, the programmable control module 370 is operatively coupled to the pump assembly 306. Specifically, in the illustrated embodiment, the programmable control module 370 is operatively coupled to the pumps 320-1 and 320-2. In some embodiments, the programmable control module 370 is configured to activate and deactivate the pumps 320-1 and 320-2. Accordingly, the programmable control module 370 is configured to active or deactivate the pumps in order to control the pressure or the state (inflated state or deflated state) of the inflatable member.

In some embodiments, the programmable control module 370 is programed to activate or deactivate the pumps 320-1 and 320-2 according to a schedule. For example, in some embodiments, the programmable control module 370 is configured to cause the inflatable member to inflate and deflate while the user is asleep. In some embodiments, the implant includes an accelerometer. The accelerometer may be configured to determine when the patient is asleep. In other embodiments, the implant includes another means or mechanism for determining when the patient is asleep. In some embodiments, the programmable control module 370 is configured to cause the inflatable member to inflate and deflate a plurality of times over a period of a few hours.

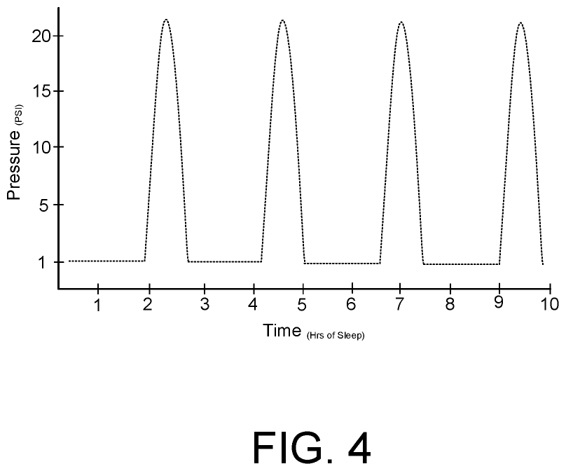

FIG. 4 is a graph of the pressure of an inflatable member of a device over a period of time. In this example, the pressure of the inflatable member increases and decreases four times over a period of 10 hours. For example, this could happen during the patient's sleep hours. In some embodiments, the programmable control module is programed or configured to cause the pressure of the inflatable member to increase and decrease a plurality of times over a course of a several hours. In the illustrated graph, the period of high pressure (or inflated state) is less than an hour and the period of time between high pressures is about 2 hours. In other embodiments, the periods of time may differ.

In some embodiments, the schedule of inflations and deflations help stretch the bodily tissue surrounding the implant. For example, after the implantation of the device, it may be helpful or beneficial to stretch the surrounding bodily tissue. The inflation and deflation cycle my help facilitate the stretching of the surrounding bodily tissue. In some cases, the stretching of the bodily tissue may be beneficial to the health of the surrounding bodily tissue.

In some cases, patients are asked to inflate and deflate the device regularly for a period of time after the implantation. In such cases, a patient or physician can program the device to inflate or deflate according to a desired schedule. In some embodiments, the schedule is set such the device inflates and deflates once a day. In other embodiments, the schedule is set such that the device is configured to inflate once a day for a period of several weeks and then may inflate and deflate more or less than once a day.

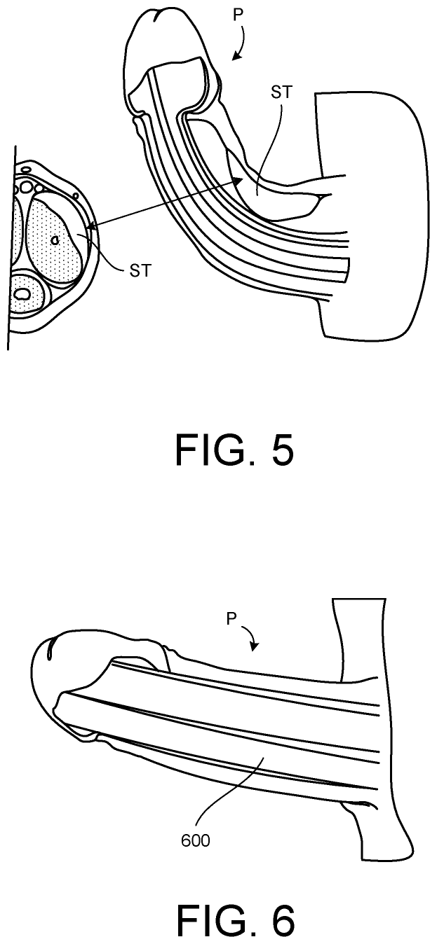

In some embodiments, the implantable inflatable device may be used to heal or repair tissue within a body of a patient. For example, in some embodiments, the implantable inflatable device may be implanted within a penis of a patient who suffers from Peyronie's disease. As illustrated in FIG. 5, Peyronie's disease is a disease state in the penis caused by a fibrous scar tissue ST or plaque that forms inside the penis P. It can result in a bent or kinked penis, particularly when the penis is in an erect state.

In some cases, an implantable inflation device is placed within a penis of a patient to help treat Peyronie's disease. For example, in a Peyronie's modelling intervention procedure, a device may be placed within a penis of a patient and can be repeatedly inflated and deflated to cause the scar tissue or plaque to dislodge, crack, or break up. In some cases, the programmable control module can be set to cause the device to inflate and deflate according to an optimal schedule to treat the disease, such as Peyronie's disease.

FIG. 6 illustrates a portion of the implantable inflation device 600 disposed within a penis P of a patient. In this figure, the scar tissue or plaque has been dislodged or otherwise removed from the tissue of the penis.

In some cases, biofilm builds up on or around devices once they have been placed within the body of a patient. This may cause the growth of microbes on or near the implanted devices. The microbes may cause an infection within the body of the patient.

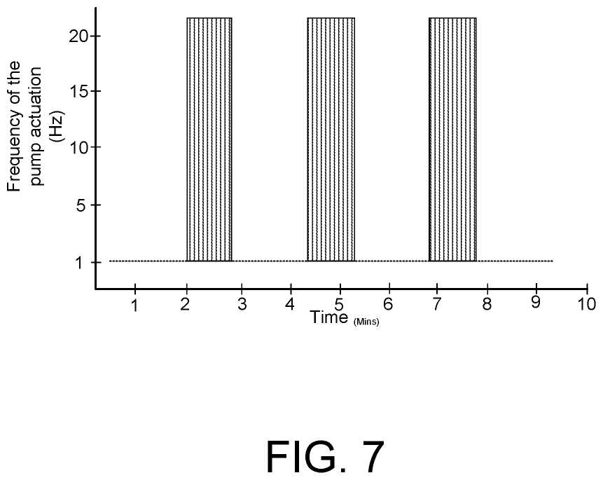

In some causes pulsing, vibrating, or quick small movements of the implanted device may help prevent or reduce the buildup of biofilm and may thus reduce the risk of infection within the body of the patient. Accordingly, in some embodiments, the programmable control module may pulse or otherwise cause the frequency of the pump actuation to increase and decrease a plurality of times over a short period of time. FIG. 7 illustrates a graph showing an example schedule of frequency changes over a period of time. In this example, the period of time is about 10 minutes. In those 10 minutes the frequency pulses or changes many times.

In some embodiments, the vibrations of the pumps cause the pulsing or vibrating of the device. For example, in some embodiments, the vibrations of the pumps transfer through the fluid to other portions, such as the inflatable members and the reservoir, to vibrate or slightly move the other portions of the device. Additionally, in some embodiments, cycling of the pumps will cause pressure oscillations in the inflatable member, leading to the inflatable members to vibrate or slightly move.

In some embodiments, the device may be set or programed to pulse during recreational activities such as intercourse.

While certain features of the described implementations have been illustrated as described herein, many modifications, substitutions, changes, and equivalents will now occur to those skilled in the art. It is, therefore, to be understood that the appended claims are intended to cover all such modifications and changes as fall within the scope of the embodiments.

What is claimed is:

1. An implantable inflatable device, comprising:
a fluid reservoir defining a cavity;
an inflatable member;
a pump assembly configured to transfer fluid from the fluid reservoir to the inflatable member; and
a programmable control module, the programmable control module being configured activate the pump assembly to transfer fluid from the fluid reservoir to the inflatable member.

2. The implantable inflation device of claim 1, wherein the programmable control module is configured to be controlled by a device located outside of a body of the patient.

3. The implantable inflation device of claim 1, wherein the programmable control module is configured to activate the pump assembly according to a schedule.

4. The implantable inflation device of claim 1, wherein the programmable control module is configured to activate the pump assembly while a user is asleep.

5. The implantable inflation device of claim 1, wherein the programmable control module is configured to activate the pump assembly such that the inflatable member inflates and deflates according to a schedule.

6. The implantable inflation device of claim 1, wherein the programmable control module is configured to activate the pump assembly such that the inflatable member inflates and deflates while a user is asleep.

7. The implantable inflation device of claim 1, wherein the programmable control module is configured to activate the pump assembly such that the inflatable member inflates and deflates according to a schedule.

8. The implantable inflation device of claim 1, wherein the programmable control module is configured to activate the pump assembly such that the inflatable member inflates and deflates a plurality of times over a period of several hours.

9. The implantable inflation device of claim 1, wherein the programmable control module is configured to activate the pump assembly such that a frequency of the pump actuation pulses over a period of time.

10. The implantable inflation device of claim 1, wherein the programmable control module is configured to activate the pump assembly such that the frequency of the pump actuation pulses a plurality of time over a period of 10 minutes.

11. A method, comprising:
activating a pump assembly of an inflation device disposed within a body of a user such that a pressure within an inflatable member of the inflation device increases and decreases a plurality of times according to a schedule.

12. The method of claim 11, wherein the pressure within the inflatable member of the inflation device increases and decreases a plurality of times while the user is asleep.

13. The method of claim 11, wherein the pressure within an inflatable member of the inflation device increases and decreases a plurality of times over a period of hours.

14. The method of claim 11, wherein the pressure within an inflatable member of the inflation device increases and decreases a plurality of times over a period of minutes.

15. The method of claim 11, wherein the inflation device is at least partially disposed within a pelvic region of the user.

16. The method of claim 11, wherein the inflation device is at least partially disposed within a penis of a user.

17. The method of claim 11, wherein the inflatable member of the inflation device is at least partially disposed within a pelvic region of the user.

18. The method of claim 11, wherein the inflatable member of the inflation device is at least partially disposed within a penis of a user.

19. The method of claim 11, wherein the inflation device includes a fluid reservoir.

20. The method of claim 11, wherein the inflation device includes a programmable control module, the programmable control module being configured activate the pump assembly.
